(19) United States
(12) Patent Application Publication
(10) Pub. No.: US 2008/0096238 A1
(43) Pub. Date: Apr. 24, 2008

(54) HIGH THROUGHPUT ASSAY FOR HUMAN RHO KINASE ACTIVITY WITH ENHANCED SIGNAL-TO-NOISE RATIO

(75) Inventors: Najam A. Sharif, Keller, TX (US); Colene D. Drace, Fort Worth, TX (US); Gary W. Williams, Burleson, TX (US)

Correspondence Address: WINSTEAD PC, P.O. BOX 50784, DALLAS, TX, 75201, US

(73) Assignee: ALCON, INC., Huenenberg (CH)

(21) Appl. No.: 11/951,992

(22) Filed: Dec. 6, 2007

Related U.S. Application Data
(63) Continuation-in-part of application No. 11/090,689, filed on Mar. 25, 2005.
(60) Provisional application No. 60/557,761, filed on Mar. 30, 2004.

(51) Int. Cl. C12Q 1/48

(52) U.S. Cl. 435/15

(57) ABSTRACT

The present invention provides a high throughput assay with increased signal-to-noise ration for human Rho kinase activity in vitro, and methods and kits therefor. A high throughput method of assaying a test compound for human Rho kinase modulating activity is also provided.

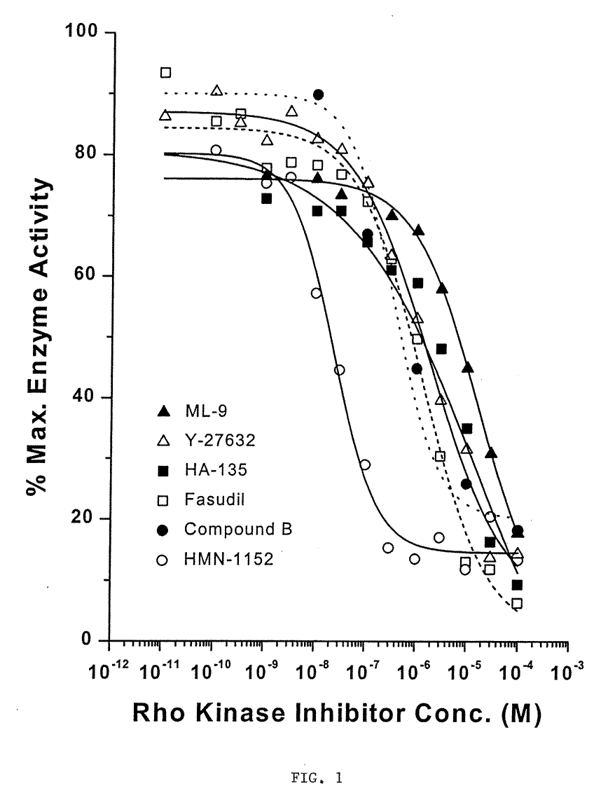

### RELATED APPLICATIONS

This application claims the benefit of U.S. Provisional Patent Application No. 60/557,761, filed Mar. 30, 2004, and is a continuation in part of U.S. application Ser. No. 11/090,689, filed Mar. 25, 2005, both of which are hereby incorporated by reference in their entirety.

### SUMMARY OF THE INVENTION

The present invention provides a high throughput method of assaying a test compound for human Rho kinase modulating activity with enhanced signal-to-noise ratio. The method comprises contacting the test compound, an agent having human Rho kinase activity, γ³³P-ATP, and a Rho kinase substrate in a medium with mixing, in a microtiter plate format, and for a time to allow phosphorylation of the substrate, thereby forming a test mixture; separating the test mixture into a first portion containing γ³³P-labeled substrate onto a filter mat and a second portion containing γ³³P-ATP using vacuum filtration and automated washing of the filter mat; drying the filter mat using microwave radiation; detecting the presence of γ³³P in the first portion; and comparing the presence of γ³³P in the first portion with presence of γ³³P-label in a first portion of a control mixture lacking the test compound.

In the above assay, a greater presence of γ³³P in the first portion of the test mixture as compared to the presence of γ³³P in the first portion of the control mixture indicates stimulatory activity of the test compound for human Rho kinase activity. Further, a lesser presence of γ³³P in the first portion of the test mixture as compared to the presence of γ³³P in the first portion of the control mixture indicates inhibitory activity of the test compound for human Rho kinase activity.

A further embodiment of the present invention is a kit for a high throughput assay of human Rho kinase activity. The kit comprises a first container means, such as a vial, ampoule, test tube, box, dish, and/or the like, comprising an agent having human Rho kinase activity, a second container means, such as a vial, ampoule, test tube, box, dish, and/or the like, comprising a Rho kinase substrate, a microtiter plate, a filter mat, and a third container means, such as a vial, ampoule, test tube, box, dish, and/or the like, comprising medium for phosphorylation of the substrate.

A method for determining Rho kinase activity presence in a test sample from a mammalian source is a further aspect of the present invention. The method comprises contacting the test sample, γ³³P-ATP, and a Rho kinase substrate in a medium with mixing, in a microtiter plate format, and for a time to allow phosphorylation of the substrate, thereby allowing formation of a γ³³P-labeled substrate mixture; separating the mixture into a first portion containing γ³³P-labeled substrate onto a filter mat and a second portion containing γ³³P-ATP using vacuum filtration and automated washing of the filter mat; drying the filter mat using microwave radiation; and detecting the presence of γ³³P-label in the first portion.

### BRIEF DESCRIPTION OF THE DRAWING

The drawing shows human recombinant ROCK II enzyme inhibition by various Rho kinase inhibitors using methods of the present invention. The symbols represent data for the following inhibitors: ▴, ML-9; Δ, γ-27632; ▪, HA-135; □, Fasudil; ●, Compound B; ◯, HMN-1152.

### DETAILED DESCRIPTION OF THE INVENTION

The present invention provides efficient and sensitive methods and compositions for detecting, identifying, or characterizing Rho kinase activity and specific inhibitors thereof using an agent having human Rho kinase activity with enhanced signal-to-noise ratio. The assay methods of the present invention provide automated and robotic procedures to render the assay into a high throughput format, provide a phosphate donating radioactive ATP ([γ-³³P]-ATP) that is safer to use than γ-³²P-ATP, provide for use of fewer hazardous chemicals in and during the assay (i.e. not using acetone which is carcinogenic), provide for mixing the reaction reagents during the assay to ensure high efficiency of substrate phosphorylation, provide for a rapid vacuum filtration to terminate the assay reactions and including automated washing of a whole 96-sample containing glass-fiber filter mat to eliminate the unused γ-³³P-ATP while retaining the phosphorylated product, and provide for rapid drying of the filter mats using microwave radiation. Further, a beta-counter that simultaneously determines the radioactivity for 8 samples at a time from the washed filter mats, and automated data capture and transfer to a computer program for automated curve-fitting of the data are performed as known to one of ordinary skill in the art in light of the present disclosure. A net fold phosphorylation of substrate by recombinant human ROCK II was found to be 4.4-fold above basal blanks for 14 studies and 42 separate assay determinations.

As used herein and unless otherwise indicated, the terms “a” and “an” are taken to mean “one”, “at least one” or “one or more”.

As used herein, “an agent having human Rho kinase activity,” means catalytic turnover by the catalytic domain of enzyme proteins referred to as Rho kinase, ROKα, ROCK II, ROCK I/ROKβ (an isoform of Rho kinase), a fusion protein of Rho kinase such as with GSK (glutathione S-transferase)-Rho kinase (6-553)-CAT (catalytic domain), or p160 ROCK, for example. The human Rho kinase activity may be in form of human recombinant Rho kinase, amino acids 11-552 of human recombinant Rho kinase (SEQ ID NO:3), amino acids 27-530 of human ROCK-1, or may be fused with a hexahistidine tag, for example. The “agent having human Rho kinase activity” has at least about 90% identity with the kinase domain of human Rho kinase. Rho kinases may be isolated using methods known to one of ordinary skill in the art, for example, methods as described by Amano et al. (Methods in Enzymology, 325: 149-155, 2000), Uehata et al. (U.S. Pat. No. 6,218,410), and Bain et al. (Biochem J., 371: 199-204, 2003).

As used herein, “a Rho kinase substrate,” means a peptide, polypeptide, or protein that accepts a phosphate group from ATP in the presence of human or mammalian Rho kinase. The Rho kinase substrate may have a sequence consisting essentially of SEQ ID NO: 1 (Long S6 Peptide from Upstate, see infra); a peptide having a sequence consisting essentially of the amino acids KKRNRTLSV, SEQ ID NO:2; a peptide having a sequence consisting essentially of the amino acids AKRRRLSSLRA, SEQ ID NO:4, a protein selected from the group of histone HI, histone H2, histone H3, and histone H4; or a protein comprising myosin basic protein, myosin binding subunit, ezrin, radizin, moesin, or adducin, for example.

The reaction conditions and medium for allowing phosphorylation of the substrate are such that kinase activity is linear with respect to time and concentration of kinase agent. Conditions and medium typically include a buffer, ATP, MgCl₂, a chelator, a reducing agent, enzyme cofactor, enzyme stabilizer, a volume from 25 microliters to 250 microliters, and/or a temperature from ambient to 37° C. For example, the buffer may be MOPS, 10 mM-100 mM, pH 7.0 to pH 7.5; MOPS, 15 mM-50 mM, pH 7.0 to pH 7.2; MOPS, 20 mM, pH 7.2; Tris/HCl, 10 mM-100 mM, pH 7.2-pH 7.7; Tris/HCl, 20 mM-50 mM, pH 7.2-pH 7.5; Tris/HCl, 50 mM, pH 7.5; or an ethanesulfonic acid buffer such as HEPES (N-2-hydroxyethylpiperazine-N′-2-ethanesulfonic acid) at 20 mM to 200 mM, pH 7.2 to pH 7.7, or at 50 mM to 100 mM, pH 7.5, for example.

Label in the form of γ-³³P-ATP is provided for the assay since the energy of the radiation particle is lower and therefore safer than that from the γ-³²P isotope. Total ATP is provided in the reaction medium in concentrations of 5 micromolar to 100 micromolar. MgCl₂ is also provided in concentrations ranging from 5 mM to 25 mM, 10 mM, or 5 mM, for example. Further components of the medium may include beta-glycerol phosphate as an enzyme stabilizer, calcium chelator EGTA, for example, reducing agent DTT or betamercaptoethanol, for example, and orthovanadate as an enzyme cofactor, for example.

The reaction is carried out with mixing to ensure optimal phosphorylation at temperatures from ambient to 37° C., or from ambient to 30° C., or at 30° C., for a time of 5 minutes to one hour, of 10 minutes to 30 minutes, or 30 minutes.

The term “modulate,” as used herein, means that the Rho kinase activity is increased or decreased in the presence of a test compound. The methods of the invention may be used to determine whether a compound is an inhibitor or stimulator of Rho kinase activity.

In various embodiments, it is desirable to increase the signal-to-noise ratio in the test. In an embodiment, pretreatment by washing with polyethyleneimine or bovine serum albumen is used to increase the signal-to-noise ratio. For washing with polyethyleneimine, the filter-mats can be soaked in a solution of 0.3% polyethyleneimine for 15 min at 23 C. in order to reduce the non-specific binding of the radioligand to the filter-mat. For washing with serum albumen, the filter-mats can be soaked in a solution of 0.1% bovine serum albumen for 15 min at 23 C. in order to reduce the non-specific binding of the radioligand to the filter-mat. Both procedures increase the signal-to-noise ratio and would thus make the assay more robust and reproducible.

Vacuum filtration and automated washing of the filter mat contributes to the high throughput efficiency of the method of the present invention. Washing may be carried out with acids such as phosphoric acid, or alcohols such as methanol or ethanol, for example. Further, drying the filter mat with microwave radiation such as with a microwave oven contributes to efficiency of detecting radioactivity.

The test compound is an inhibitor of Rho kinase activity if the amount of radioactive label in the first portion of the test mixture is lower than the amount of radioactive label present in the first portion of a control mixture lacking the test compound. The test compound is identified as a Rho kinase activity inhibitor if the amount of radioactive label in the first portion of the test mixture is less than 90% of the activity of the Rho kinase in a control mixture lacking the test compound. In other embodiments of the present invention, the test compound is identified as a Rho kinase activity inhibitor if the amount of radioactive label in the first portion of the test mixture is less than 80%, 70%, 60%, 50%, 40%, 30%, 20% or 10% of the activity of the Rho kinase in a control mixture lacking the test compound.

The test compound is a stimulator of Rho kinase activity if the amount of radioactive label in the first portion of the test mixture is greater than the amount of radioactive label present in the first portion of a control mixture lacking the test compound. The test compound is identified as a Rho kinase activity stimulator if the amount of radioactive label in the first portion of the test mixture is greater than 110% of the activity of the Rho kinase in a control mixture lacking the test compound. In other embodiments of the present invention, the test compound is identified as a Rho kinase activity stimulator if the amount of radioactive label in the first portion of the test mixture is greater than 120%, 130%, 140%, 150%, or more of the activity of the Rho kinase in a control mixture lacking the test compound.

A test sample from a mammalian source means a sample of blood, plasma, tissue, urine, body secretion, swab, or extract from a mammal.

The methods of the invention are suitable for high throughput screening, i.e. screening of large numbers of candidate Rho kinase modulators for generating leads to pharmaceutical products. In such screening assays, compounds may be put into groups for screening using microtiter plate technologies. The methods of the invention are performed in small volumes associated with 384 and 1536 well plates, in addition to the 96 well plate format. Each well has a small volume, usually 250 to 300 microliters in a 96 well plate, 60 to 70 microliters in a 384 well plate and 6-8 microliters in a 1536 well plate. In the high throughput assays of the invention, it is possible to screen up to several thousand different modulators in a single day. Each well of a microtiter plate can be used to run a separate assay against a test compound, or, if concentration or incubation time effects are to be tested, every 5-10 wells can test a single candidate test compound. Therefore, one standard microtiter plate can assay 96 modulators.

The test compounds may be any small chemical compound, or a biological compound, such as a protein, carbohydrate, nucleic acid or lipid. Test compounds are dissolved in aqueous or organic solutions (e.g., ethanol, methanol, DMSO, or a mixture of organic solvents, for example). The high throughput screening methods involve providing a candidate test compound, a combinatorial chemical library, a peptide library, or the like, for screening for Rho kinase modulator activity.

A kit of the present invention as set forth herein may further comprise a fourth container means comprising γ-³³P-ATP. Alternatively, or in addition, a kit may further comprise a control compound having inhibitory activity for human Rho kinase activity. Exemplary inhibitory compounds are listed in Table 1. The medium of the kit may conveniently comprise one or more buffers for reconstituting, diluting or dissolving the kinase, substrate and/or ATP. The kit may also further comprise a reagent for stopping the reaction by washing, for example, phosphoric acid.

### EXAMPLES

Protein kinases represent one of the largest group of enzymes having activity in the modulation of a wide variety of cellular events connected with signal transduction processes. These enzymes act by transferring phosphate groups to amino acids of other intracellular polypeptides/proteins to either activate or inhibit the activity of these proteins. Such phosphorylating actions of protein kinases are involved in many diverse down-stream cellular functions such as blood vessel relaxation, and hormone release, for example.

Rho kinases represent a family of serine threonine kinase enzymes that are powered by Rho-activated phosphorylation. Rho kinases are also known as Rho-associated coiled-coil-forming protein kinases (ROCK). ROCK I and ROCK II are isoforms of Rho kinases that have now been cloned from many species and the sequences deposited in the Genbank database. The cloned human Rho kinase (ROCK II; Genbank sequence gi 4759044) is composed of 1388 amino acids while the cloned rat ROCK II (Genbank sequence gi 6981478; Accession number Genbank 3327051) is composed of 1379 amino acids. The cloned human ROCK II and cloned rat ROCK II enzymes share 85% homology based on their protein sequences, indicating potentially significant species differences. Cloned bovine ROCK I (Genbank sequence gi 27806123) and cloned human ROCK II also share 87% homology despite their identical length of 1388 residues.

Rho activity is primarily regulated by Rho-specific guanine nucleotide exchange factors. Effectors for Rho include Rho, Ras, TC10 and Cdc42. This kinase family controls the organization of the actin cytoskeleton. Targets for Rho include myosin light chain, myosin light chain kinase (MLCK) and myosin phosphatase; all enzymes involved in inducing smooth muscle contraction. Rho kinase signaling pathway has been linked to numerous cellular functions such as differentiation, cell and/or tissue contraction or relaxation, transmitter/hormone secretion, motility, adhesion and growth, for example. Rho kinase signaling pathway has been implicated in various diseases including systemic hypertension, vasospasm, bronchial asthma, progression of atherosclerosis, cancer, erectile dysfunction and glaucoma (J. Mol Med. 80: 629-638, 2002).

Rho kinase activity has been monitored using a number of different techniques using Western blot analysis and polyclonal antibodies to the Rho kinase target protein (Uehata et al. Nature 389: 990-994, 1997; Rao et al. Invest Opthalmol Vis. Sci. 42: 1029-1037, 2001); using phosphorylation of MLCK or other substrates with [γ-³²P]-ATP followed by liquid scintillation counting of the phosphorylated target protein isolated using Whatman P81 filter paper washed manually with phosphoric acid (Amano et al. J. Biol. Chem. 274: 32418-32424, 1999); and using phosphorylation of histone HI using [γ-³²P]-ATP followed by liquid scintillation counting of the phosphorylated target protein isolated using a centrifugation assay (Nagumo et al. Am. J. Physiol Cell Physiol. 278: C57-C65, 2000). A method of utilizing recombinant rat Rho kinase (ROCK II) was described in Bain et al. (Biochem. J. 371: 199-204, 2003) and Davies et al., (Biochem. J. 351: 95-105, 2000).

Variations of the methods mentioned above include a chemiluminescence assay (PCT Published Patent Application No. WO 02/085909); [γ-³³P]-ATP-linked phosphorylation of myelin basic protein followed by P30 membrane-based isolation of the phosphorylated product and liquid scintillation counting (PCT Published Patent Application No. WO 02/076977); using [γ-³²P]-ATP and Rho kinase isolated from bovine aorta and from human platelets and using a membrane filter procedure to isolate the phosphorylated product (U.S. Pat. No. 6,218,410) and; as for the latter procedure, and also using SDS-PAGE followed by cutting out of the gel bands containing the phosphorylated histone product and counting (U.S. Pat. No. 6,451,825). Other more generic protein kinase assays (but not for Rho kinases) have been reported where the kinase activity is monitored using a bioluminescence assay for the ATP used in the reaction (U.S. Pat. No. 6,599,711); and also colorimetric assays and other variations of the afore-mentioned assays (e.g. U.S. Pat. No. 5,759,787).

Upstate USA Inc. (Charlotteville, Va.) sells human recombinant Rho kinase (ROCK II) and a substrate, and provides an assay protocol using these reagents. The Upstate USA Inc. assay method uses [γ-³²P]-ATP, a fast-decaying high energy dangerous radioisotope The reaction is run and terminated manually followed by spotting of reaction mixture aliquots on individual P81 filter paper squares, manually rinsing the squares in phosphoric acid, drying the squares in acetone, manually transferring the paper squares into scintillation vials, adding scintillation fluid, and counting the radioactivity in each vial on a beta-counter. The Upstate USA Inc assay is a low throughput assay, is not automated, is relatively unsafe since it uses many hazardous chemicals (e.g. [γ-³²P]-ATP and acetone), is laborious and time-consuming by virtue of the reagents and procedures recommended in their assay kit. Further, manual rinsing of individual filter paper squares contributes to lack of sensitivity and reproducibility expected of such an assay.

### Example 1

### In vitro High Throughput Assays for Determination of Rho Kinase Activity and Modulation Thereof

Human recombinant Rho kinase (ROKα/ROCK-II, (amino acids 11-552, SEQ ID NO:3), human active, catalog #14-451, Upstate USA, Inc., Lake Placid, N.Y.), MgCl₂/ATP cocktail, and enzyme substrate (all from Upstate) are used in the present assay. The enzyme assays are performed using a Biomek 2000 Robotic Workstation (Beckman Instruments, Palo Alto, Calif.) in a 96-well format using γ-³³P-ATP (Perkin-Elmer Life Sciences, Boston, Mass.). Stock γ-³³P-ATP (3000 Ci/mmol) is diluted to 1 μCi/μl with the MgCl₂/ATP cocktail solution. The concentrations of MgCl₂/ATP used are 15 mM and 100 μM, respectively. The ROKα/ROCK-II (human, active, 1 ng per well) is assayed using the Long S6 substrate peptide (32 amino-acid; KEAKEKRQEQIAKRRRLSSLRASTSKSGGSQK, SEQ ID NO:1, (30 μM final) (from Upstate USA Inc.). The substrate and enzyme are diluted in 20 mM MOPS buffer (pH 7.2), 25 mM β-glycerol phosphate, 5 mM EGTA, 1.0 mM sodium orthovanadate, and 1.0 mM dithiothreitol. Test compound dilutions are made in 10:10 dimethyl sulfoxide-ethanol (vol/vol). In the following order, substrate, enzyme, test compound dilution, and [γ-³³P]-ATP are added to the 96-well plates for a final volume of 100 μp per well. The 96-well plates are then placed on a slow speed rotary mixer (Roto Mix; THERMOLYNE® from VWR, Dallas, Tex.) in an incubator set to 30° C. to gently mix the reagents during the assay to ensure efficient substrate phosphorylation. After an incubation of 30 min at 30° C., the assays are terminated by rapid simultaneous aspiration of the reaction mixtures from each of the 96-wells onto a pre-wetted negatively-charged P30 glass filter mat (Wallac Inc., Turku, Finland) by vacuum filtration using a cell harvester (Mach II; TomTec, Hamden, Conn.) followed by rapid automated washing of each sample area of the filter mat with 3×7 ml of 0.75% phosphoric acid (23° C.). The unutilized [γ-³³P]-ATP and other residual reagents are thereby eliminated from the filter mat but the radioactive phosphorylated peptide product is retained on the filter mat for quantification. The automated washing of the filter mat thereby enhances the signal-to-noise ratio of the assay thus rendering it into a sensitive assay. The filter mats bearing the captured radioactive phosphorylated product are then dried in a microwave oven for 15 sec, placed in special sample cellophane bag (Wallac Inc., Turku, Finland) designed for these filter mats and covered with 20 ml of Betaplate scintillation fluid (Perkin-Elmer Inc., Boston, Mass.). The bags are then sealed using a heat sealer device (Wallac Inc., Turku, Finland) and a roller used to evenly spread the scintillation fluid over the whole of the filter mat. The radioactivity captured on the filter mats is then determined on a 1205-Betaplate (Wallac Inc., Turku, Finland) beta-scintillation-counter that measures 8 samples simultaneously, counting each sample for 1 minute.

In a further embodiment of the present invention, the dried filter mat can be covered with a solid scintillant (MELTILEX®; Wallac Inc., Turku, Finland) that is melted directly onto the filter mat using a MELTILEX® heat sealer (Wallac Inc., Turku, Finland), a device designed for this purpose. The coated filter mat is then placed in a sample cellophane bag (Wallac Inc., Turku, Finland) designed for these filter mats that is then sealed and the radioactivity determined as described above. The solid scintillant assists in avoiding the spread of radioactivity from sample to sample on the dried filter mat and thus minimizes variation of the data. A solid scintillant also eliminates waste disposal of liquid scintillation fluid.

The raw data from the filter mats are then automatically sent electronically to a computer for semi-automated analyses using algorithms and a suite of programs (XLFITT computer program; IDBS corporation, Emeryville, Calif.) that perform non-linear, iterative, sigmoidal-fit analyses of the raw data. The test compound potencies for inhibiting the human recombinant ROCK II enzyme activity are then generated and tables of data and appropriate graphs constructed as previously described (Sharif et al., J. Pharmacol Exp. Ther. 286:1094-1102, 1998; Sharif et al., J Pharmacol. Expt. Ther. 293:321-328, 2000; Sharif et al. J. Ocular Pharmacol Ther. 18:141-162, 2002a; Sharif et al. J. Pharmac. Pharmacol. 54:539-547, 2002b).

The recombinant human ROCK II enzyme inhibition constants for various compounds shown in Table 1 below are the IC₅₀ values (the concentration of the compound that inhibits the enzyme activity by 50% of the maximum) determined as previously described (Sharif et al., ibid.). The drawing depicts representative enzyme inhibition curves to illustrate the type of data that can be generated from such assays to determine the recombinant human ROCK II inhibitory potency of various compounds. Table 1 shows the structures of cited compounds and their relative potencies at inhibiting human recombinant ROCK II enzyme activity as determined from several experiments using the assay procedures described above.

TABLE 1
Enzyme Inhibition Constants (IC₅₀) Obtained for Various Compounds Against
Human Recombinant ROCK II Enzyme

| | | Inhibition Constant |
|---|---|---|
| | | (IC₅₀, nM) & Hill |
| Compound | Chemical Structure of Compound | Coefficient (nH) |
| A, HMN-1152 | 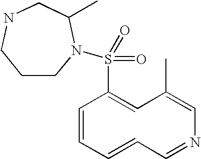 | 47 ± 14 nM (N = 4) (nH = 0.99 ± 0.12) |
| B | 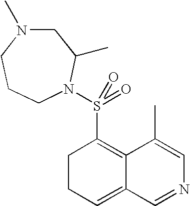 | 485 ± 207 nM (N = 3) (nH = 0.6 ± 0.2) |
| C | 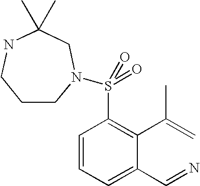 | 1512 ± 704 nM (N = 4) (nH = 0.88 ± 0.21) |
| D, Fasudil | 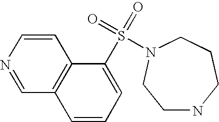 | 1690 ± 185 nM (N = 10) (nH = 0.91 ± 0.06) |
| E, H-7 | 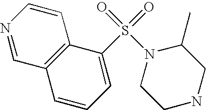 | 2341 ± 395 nM (N = 5) (nH = 0.99 ± 0.13) |
| F | 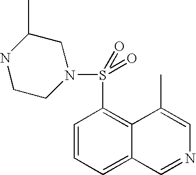 | 2625 ± 307 nM (N = 4) (nH = 0.94 ± 0.11) |
| G, Y-27632 | 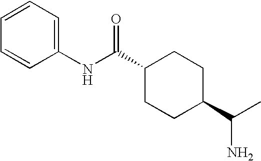 | 2802 ± 865 nM (N = 3) (nH = 0.91 ± 0.19) |
| H | 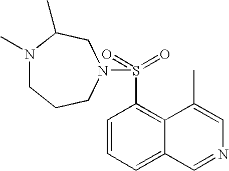 | 3463 ± 1800 nM (N = 4) (nH = 0.95 ± 0.16) |
| I, HA-135 | 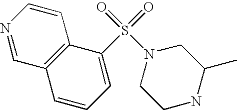 | 6702 ± 900 nM (N = 2) (nH = 0.91 ± 0.28) |
| J, ML-9 | 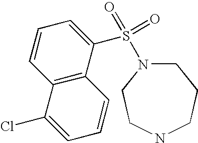 | 12003 ± 995 nM (N = 2) (nH = 0.9 ± 0.3) |

Data are mean ±SEM; N=the number of assays conducted and nH=Hill coefficient of the inhibition plots. Note that the Hill coefficients are close to unity for most of the compounds indicating a monophasic inhibition of the active site of the ROCK II enzyme.

The data shown in Table 1 indicate that Rho kinase activity can be differentially inhibited by the cited compounds.

Data from this set of assay procedures for quantifying recombinant human ROCK II enzyme activity exemplify the sensitivity and reproducibility of the assay as follows.
Total [γ-³³P]-ATP DPM (disintegrations per min) added to reaction mixture=2,465,8391±665,190 (n=14)
Maximum DPMs found in phosphorylated substrate (i.e. product)=52,557±2,058 (n=14) (0.22% of total added DPMs)

Substrate blank DPMs=12,156±598 (n=14) (0.049% of total added DPMs)

Net fold phosphorylation of substrate by recombinant human ROCK II=4.4-fold above basal blanks (n=14 experiments; 42 separate assay determinations).

### Example 2

### In Vitro Assay Results Inversely Correlate With In Vivo Results

Data such as that of Table 1 are then used to rank order compounds based on the degree of inhibition of recombinant human ROCK II enzyme and also used to select compounds for further testing to determine their functional inhibitory activity (for their ability to lower intraocular pressure (IOP) in rabbits and ocular hypertensive monkeys or to relax pre-contracted blood vessels in organ baths or for them to increase blood flow in vivo in various laboratory animals, for example).

The data shown in Table 2 are the average IOP reductions at each time point from 7-8 rabbits for drug-treated and vehicle-treated groups.

TABLE 2
Intraocular Pressure Reducing Effects of Rho Kinase Inhibitors
In Rabbit Eyes Illustrate Usefulness and Correlation with
In Vitro Recombinant Human ROCK II Data

| | | | % Max. IOP Reduction in | | | |
|---|---|---|---|---|---|---|
| | | | Rabbit Eyes | | | |
| | Topical | | 1 Hour | 2 Hours | 3 Hours | 4 Hours |
| | Ocular | | Post | Post | Post | Post |
| Compound | Dose (μg) | | Dosing | Dosing | Dosing | Dosing |
| A, HMN-1152 | 500 | μg | 34 | 34 | 27 | 19 |
| (ROCK II | 1 | mg | 29 | 35 | 32 | 24 |
| IC₅₀ = 47 nM) | | | | | | |
| D, Fasudil | 300 | μg | 17 | 29 | 26 | 20 |
| (ROCK II | 500 | μg | 25 | 33 | 25 | 17 |
| IC₅₀ = 1690 nM) | | | | | | |
| E, H-7 | 500 | μg | 19 | 11 | 0 | 6 |
| (ROCK II | | | | | | |
| IC₅₀ = 2341 nM) | | | | | | |
| G, Y-27632 | 2 | mg | 26 | 28 | 29 | 27 |
| (ROCK II | | | | | | |
| IC₅₀ = 2802 nM) | | | | | | |
| J, ML-9 | 300 | μg | 8 | 23 | 0.4 | 6 |
| (ROCK II | | | | | | |
| IC₅₀ = 12,003 nM) | | | | | | |

The vehicle caused insignificant IOP changes in the dosed and un-dosed eyes. The data of Table 2 show that the inhibitor potency is inversely related to the IC₅₀ value, the ROCK II enzyme inhibitory potency reflects closely the IOP-lowering activity of the compounds and, therefore, the in vitro recombinant human ROCK II enzyme assay predicts in vivo efficacy of the Rho kinase inhibitors tested.

The data shown in Table 3 are the average IOP reductions at each time point from 8 monkeys for the drug treatment relative to 5 monkeys for the vehicle-treated group.

TABLE 3
Intraocular Pressure Reducing Effects of Rho Kinase Inhibitors
in the Conscious Ocular Hypertensive Cynomolgus Monkey
Eyes Illustrates Usefulness and Correlation with the
In Vitro Recombinant Human ROCK II Data

| | | | % Max. IOP Reduction in Ocular | | |
|---|---|---|---|---|---|
| | | | Hypertensive Monkey Eyes | | |
| | Topical | | 1 Hour | 3 Hours | 6 Hours |
| | Ocular | | Post | Post | Post |
| Compound | Dose (μg) | | Dosing | Dosing | Dosing |
| A, HMN-1152 | 100 | μg | 28 | 25 | 19 |
| (ROCK II | 300 | μg | 29 | 30 | 21 |
| IC₅₀ = 47 nM) | 1 | mg | 42 | 51 | 36 |
| D, Fasudil | 500 | μg | 33 | 28 | 16 |
| (ROCK II | | | | | |
| IC₅₀ = 1690 nM) | | | | | |
| E, H-7 | 1 | mg | 31 | 21 | 12 |
| (ROCK II | | | | | |
| IC₅₀ = 2341 nM) | | | | | |
| G, Y-27632 | 300 | μg | 15 | 14 | 8 |
| (ROCK II | 1 | mg | 24 | 36 | 32 |
| IC₅₀ = 2802 nM) | | | | | |
| I, HA-135 | 500 | μg | 19 | 15 | 1 |
| (ROCK II | | | | | |
| IC₅₀ = 6702 nM) | | | | | |
| F | 500 | μg | 23 | 20 | 20 |
| (ROCK II | | | | | |
| IC₅₀ = 2625 nM) | | | | | |
| J, ML-9 | 300 | μg | 7 | 6 | 5 |
| (ROCK II | | | | | |
| IC₅₀ = 12,000 nM) | | | | | |

The vehicle typically produced IOP-lowering of 6% and 14% at 3 and 6 hours post-dosing in the ocular hypertensive eyes and had minimal effect on the IOP of the untreated contralateral eyes. The ROCK II enzyme inhibitory potency reflects closely the IOP-lowering activity of the compounds, i.e. the in vitro recombinant human ROCK II enzyme assay predicts in vivo efficacy of the Rho kinase inhibitors tested.

### Example 3

### Prophetic

### In vitro High Throughput Assays for Determination of Rho Kinase Activity and Modulation Thereof

Human recombinant Rho kinase (ROKα/ROCK-II, (amino acids 11-552, SEQ ID NO:3), human active, catalog #14-451, Upstate USA, Inc., Lake Placid, N.Y.), MgCl₂/ATP cocktail, and enzyme substrate (all from Upstate) are used in this prophetic example. The enzyme assays are capable of being performed using a Biomek 2000 Robotic Workstation (Beckman Instruments, Palo Alto, Calif.) in a 96-well format using γ-³³P-ATP (Perkin-Elmer Life Sciences, Boston, Mass.). Stock γ-³³P-ATP (3000 Ci/mmol) is diluted to 1 μCi/μl with the MgCl₂/ATP cocktail solution. The concentrations of MgCl₂/ATP used are 15 mM and 100 μM, respectively. The ROKα/ROCK-II (human, active, 1 ng per well) is assayed using the Long S6 substrate peptide (32 amino-acid; KEAKEKRQEQIAKRRRLSSLRASTSKSGGSQK, SEQ ID NO: 1, (30 μM final) (from Upstate USA Inc.). The substrate and enzyme are diluted in 20 mM MOPS buffer (pH 7.2), 25 mM β-glycerol phosphate, 5 mM EGTA, 1.0 mM sodium orthovanadate, and 1.0 mM dithiothreitol. Test compound dilutions are made in 10:10 dimethyl sulfoxide-ethanol (vol/vol). In the following order, substrate, enzyme, test compound dilution, and [γ-³³P]-ATP are added to the 96-well plates for a final volume of 100 μp per well. The 96-well plates are then placed on a slow speed rotary mixer (Roto Mix; THERMOLYNE® from VWR, Dallas, Tex.) in an incubator set to 30° C. to gently mix the reagents during the assay to ensure efficient substrate phosphorylation. After an incubation of 30 min at 30° C., the assays are terminated by rapid simultaneous aspiration of the reaction mixtures from each of the 96-wells onto a pre-wetted negatively-charged P30 glass filter mat (Wallac Inc., Turku, Finland) by vacuum filtration using a cell harvester (Mach II; TomTec, Hamden, Conn.). The glass filter mat is pretreated by washing with 0.3% polyethyleneimine or bovine serum albumen. For washing with polyethyleneimine, the filter-mats can be soaked in a solution of 0.3% polyethyleneimine for 15 min at 23 C. in order to reduce the non-specific binding of the radioligand to the filter-mat. For washing with serum albumen, the filter-mats can be soaked in a solution of 0.1% bovine serum albumen for 15 min at 23 C. in order to reduce the non-specific binding of the radioligand to the filter-mat. Both procedures increase the signal-to-noise ratio and would thus make the assay more robust and reproducible.

The washing of the filter mats to increase signal-to-noise ratio is followed by rapid automated washing of each sample area of the filter mat with 3×7 ml of 0.75% phosphoric acid (23° C.). The unutilized [γ-³³P]-ATP and other residual reagents are thereby eliminated from the filter mat but the radioactive phosphorylated peptide product is retained on the filter mat for quantification. The automated washing of the filter mat thereby enhances the signal-to-noise ratio of the assay thus rendering it into a sensitive assay. The filter mats bearing the captured radioactive phosphorylated product are then dried in a microwave oven for 15 sec, placed in special sample cellophane bag (Wallac Inc., Turku, Finland) designed for these filter mats and covered with 20 ml of Betaplate scintillation fluid (Perkin-Elmer Inc., Boston, Mass.). The bags are then sealed using a heat sealer device (Wallac Inc., Turku, Finland) and a roller used to evenly spread the scintillation fluid over the whole of the filter mat. The radioactivity captured on the filter mats is then determined on a 1205-Betaplate (Wallac Inc., Turku, Finland) beta-scintillation-counter that measures 8 samples simultaneously, counting each sample for 1 minute.

In a further embodiment of the present invention, the dried filter mat can be covered with a solid scintillant (MELTILEX®; Wallac Inc., Turku, Finland) that is melted directly onto the filter mat using a MELTILEX® heat sealer (Wallac Inc., Turku, Finland), a device designed for this purpose. The coated filter mat is then placed in a sample cellophane bag (Wallac Inc., Turku, Finland) designed for these filter mats that is then sealed and the radioactivity determined as described above. The solid scintillant assists in avoiding the spread of radioactivity from sample to sample on the dried filter mat and thus minimizes variation of the data. A solid scintillant also eliminates waste disposal of liquid scintillation fluid.

The raw data from the filter mats are then automatically sent electronically to a computer for semi-automated analyses using algorithms and a suite of programs (XLFITT computer program; IDBS corporation, Emeryville, Calif.) that perform non-linear, iterative, sigmoidal-fit analyses of the raw data. The test compound potencies for inhibiting the human recombinant ROCK II enzyme activity are then generated and tables of data and appropriate graphs constructed as previously described (Sharif et al., J. Pharmacol Exp. Ther. 286:1094-1102, 1998; Sharif et al., J. Pharmacol. Expt. Ther. 293:321-328, 2000; Sharif et al., J. Ocular Pharmacol Ther. 18:141-162, 2002a; Sharif et al., J. Pharmac. Pharmacol. 54:539-547, 2002b).

The recombinant human ROCK II enzyme inhibition constants for various compounds shown in Table 1 below are the IC₅₀ values (the concentration of the compound that inhibits the enzyme activity by 50% of the maximum) determined as previously described (Sharif et al., ibid.). The drawing depicts representative enzyme inhibition curves to illustrate the type of data that can be generated from such assays to determine the recombinant human ROCK II inhibitory potency of various compounds. Table 1 shows the structures of cited compounds and their relative potencies at inhibiting human recombinant ROCK II enzyme activity as determined from several experiments using the assay procedures described above.

Although the foregoing invention and the methods associated with it have been described in some detail by way of illustration and example for purposes of clarity of understanding, it will be readily apparent to those of ordinary skill in the art in light of the teachings of this invention that certain changes and modifications may be made thereto without departing from the spirit or scope of the described procedures and claims. Those of ordinary skill in the art, in light of the present disclosure, will appreciate that modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

The references cited herein, to the extent that they provide exemplary procedural or other details supplementary to those set forth herein, are specifically incorporated by reference.

What is claimed is:

1. A high throughput method of assaying a test compound for human Rho kinase modulating activity comprising:
forming a test mixture by contacting and mixing the test compound with an agent having human Rho kinase activity, a γ³³P-ATP, and a Rho kinase substrate,
phosphorylating said test mixture in a microtiter plate format;
increasing signal-to-noise ratio of the radioligand as compared to the background by reducing the non-specific binding of the radioligand to the filter-mat;
separating the test mixture into a first portion containing γ³³P-labeled substrate onto a filter mat and a second portion containing γ ³³P-ATP using vacuum filtration and automated washing of the filter mat;
drying the filter mat;
detecting the presence of γ³³P in the first portion; and
comparing the presence of γ³³P in the first portion with presence of γ³³P-label in a first portion of a control mixture lacking the test compound,
wherein a greater presence of γ³³P in the first portion of the test mixture as compared to the presence of γ³³P in the first portion of the control mixture indicates stimulatory activity of the test compound for human Rho kinase activity; and
wherein a lesser presence of γ³³P in the first portion of the test mixture as compared to the presence of γ³³P in the first portion of the control mixture indicates inhibitory activity of the test compound for human Rho kinase activity.

2. The method of claim 1, wherein the Rho kinase substrate comprises a peptide having a sequence consisting essentially of SEQ ID NO: 1.

3. The method of claim 1, wherein the Rho kinase substrate comprises a peptide having a sequence consisting essentially of SEQ ID NO:2.

4. The method of claim 1, wherein the Rho kinase substrate comprises histone H1-H4.

5. The method of claim 1, wherein the Rho kinase substrate comprises myosin basic protein.

6. The method of claim 1, wherein the agent having human Rho kinase activity comprises human recombinant Rho kinase.

7. The method of claim 1, wherein the agent having human Rho kinase activity comprises amino acids 11-552 of human recombinant Rho kinase having SEQ ID NO:3.

8. The method of claim 1, wherein the agent having human Rho kinase activity comprises a fusion protein.

9. The method of claim 8, wherein the agent having human Rho kinase activity comprises a fusion with a hexahistidine tag.

10. The method of claim 8, wherein the agent having human Rho kinase activity comprises a fusion with GST.

11. The method of claim 1, wherein the agent having human Rho kinase activity comprises p160ROCK.

12. A high throughput method of assaying a test compound for human Rho kinase modulating activity comprising:
forming a test mixture by contacting and mixing the test compound with an agent having human Rho kinase activity, a γ³³P-ATP, and a Rho kinase substrate comprising SEQ ID NO: 1,
phosphorylating said test mixture in a microtiter plate format;
increasing signal-to-noise ratio of the radioligand as compared to the background by reducing the non-specific binding of the radioligand to the filter-mat;
separating the test mixture into a first portion containing γ³³P-labeled substrate onto a filter mat and a second portion containing γ³³P-ATP using vacuum filtration and automated washing of the filter mat;
drying the filter mat;
detecting the presence of γ³³P in the first portion; and
comparing the presence of γ³³P in the first portion with presence of γ³³P-label in a first portion of a control mixture lacking the test compound,
wherein a greater presence of γ³³P in the first portion of the test mixture as compared to the presence of γ³³P in the first portion of the control mixture indicates stimulatory activity of the test compound for human Rho kinase activity; and
wherein a lesser presence of γ³³P in the first portion of the test mixture as compared to the presence of γ33ₚ in the first portion of the control mixture indicates inhibitory activity of the test compound for human Rho kinase activity.

13. A kit for a high throughput assay of human Rho kinase activity comprising:
a first container having an agent having human Rho kinase activity,
a second container having a Rho kinase substrate,
a microtiter plate,
a filter mat, and
a third container having medium for phosphorylation of the substrate.

14. The kit of claim 13, further comprising a fourth container having γ³³P-ATP.

15. The kit of claim 13, further comprising a fourth container having a control compound having inhibitory activity for human Rho kinase activity.

16. The method of claim 1, wherein the Rho kinase substrate comprises a peptide having a sequence consisting essentially of SEQ ID NO:4.
